(19) United States
(12) Patent Application Publication
(10) Pub. No.: US 2011/0052603 A1
(43) Pub. Date: Mar. 3, 2011

(54) METHOD OF TREATMENT AND AGENTS USEFUL FOR SAME

(75) Inventors: Philippe Bouillet, Victoria (AU); Andreas Strasser, Victoria (AU); Steve Gerondakis, Victoria (AU); Fook Lin Tai, Victoria (AU); Thomas Kaufmann, Victoria (AU); Raffi Gugasyan, Victoria (AU)

(73) Assignee: THE WALTER AND ELIZA HALL INSTITUTE OF MEDICAL RESEARCH, Parkville ,Victoria (AU)

(21) Appl. No.: 12/444,579

(22) PCT Filed: Oct. 5, 2007
(86) PCT No.: PCT/AU2007/001505

§ 371 Date: Dec. 8, 2009

Related U.S. Application Data
(60) Provisional application No. 60/849,864, filed on Oct. 6, 2006.

(51) Int. Cl. A61K 39/395; C12N 5/00; A61K 31/7088; A61K 38/02; A61K 38/45; A61K 38/46; A61K 38/48; A61P 37/06; A61P 31/12; A61P 1/16; A61P 35/00

(52) U.S. Cl. 424/172.1; 435/375; 514/44 R; 514/18.9; 424/94.5; 424/94.6; 424/94.65; 424/94.64; 514/44 A

(57) ABSTRACT

The present invention relates generally to a method of modulating tumor necrosis factor-mediated apoptosis and to agents useful for same. More particularly, the present invention contemplates a method of modulating tumor necrosis factor-mediated hepatocyte apoptosis by modulating an intracellular Bim and/or Bid-dependent signalling mechanism. The method of the present invention is useful, inter alia, in the treatment and/or prophylaxis of conditions characterized by aberrant, unwanted or otherwise inappropriate tumor necrosis factor-mediated apoptosis. The present invention is further directed to methods for identifying and/or designing agents capable of modulating the subject Bim and/or Bid-dependent signalling mechanism.

### FIELD OF THE INVENTION

The present invention relates generally to a method of modulating tumour necrosis factor-mediated apoptosis and to agents useful for same. More particularly, the present invention contemplates a method of modulating tumor necrosis factor-mediated hepatocyte apoptosis by modulating an intracellular Bim and/or Bid-dependent signalling mechanism. The method of the present invention is useful, inter alia, in the treatment and/or prophylaxis of conditions characterised by aberrant, unwanted or otherwise inappropriate tumour necrosis factor-mediated apoptosis. The present invention is further directed to methods for identifying and/or designing agents capable of modulating the subject Bim and/or Bid-dependent signalling mechanism.

### BACKGROUND OF THE INVENTION

Bibliographic details of the publications referred to by author in this specification are collected at the end of the description.

The reference to any prior art in this specification is not, and should not be taken as, an acknowledgment or any form of suggestion that that prior art forms part of the common general knowledge in Australia.

Cell death may occur either via necrotic mechanisms or by controlled intracellular processes, termed apoptosis, which are characterised by a condensation and subsequent fragmentation of the cell nucleus. As such, it is a process of the deliberate relinquishment of viability by a cell in a multicellular organism. Apoptosis is usually carried out in an ordered process that confers advantages during an organism's life cycle. For example, the differentiation of human fingers in a developing embryo requires the cells between the fingers to initiate apoptosis so that the fingers can separate. However, defective apoptotic processes have been implicated in an extensive variety of diseases. Excessive apoptosis causes cell-loss disorders, whereas insufficient apoptosis results in uncontrolled cell accumulation promoting development of neoplasias. Apoptosis therefore occurs in the context of a range of both normal and pathological processes.

A cell undergoing apoptosis undergoes a characteristic and controlled process which can be summarised as follows:
(i) Subsequently to initiation of the apoptotic process by a specific signal, the cell becomes rounded. This occurs because the structural protein which form the cytoskeleton are digested by caspases which have been activated within the cell.
(ii) The chromatin undergoes initial degradation and condensation.
(iii) The chromatin undergoes further condensation into compact patches against the nuclear envelope. At this stage, the membrane surrounding the nucleus still appears complete. However, specialized caspases have already advanced in the degradation of nuclear pore proteins and have begun to degrade the lamin that underlies the nuclear envelope.
(iv) The nuclear envelope becomes discontinuous and the DNA inside it is fragmented (a process referred to as karyorrhexis). The nucleus breaks into several discrete chromatin bodies or nucleosomal units due to the degradation of DNA.
(v) Plasma membrane blebbing occurs.
(vi) The cell is phagocytosed or the cell breaks apart into several vesicles termed apoptotic bodies, which are then phagocytosed.

A diverse group of signals, as exemplified in the following table, are thought to induce apoptosis.

Apoptosis inducing signals

| | PHYSICAL | | | | | |
|---|---|---|---|---|---|---|
| CHEMICALS | INSULTS | VIRUSES | CELLS | CYTOKINES | WITHDRAWAL FROM TROPHIC FACTORS | OTHERS |
| Chemotherapeutic | Neutrons | HIV-1 | Cytolytic | TNF-α | Glucose | Glucocorticoids |
| agents | X-rays | Sindbis | T cells | TGF-β | Growth factors (Interleukin-2, Interleukin-3, | p53 |
| Glucocorticoids | β-rays | Baculo | | | Interleukin-10, Interleukin-13, Granulocyte- | c-myc |
| Free-radicals | Gamma-rays | | | | macrophage colony stimulating factor, | Ced-2, 3, 4 |
| Glutamate | UV-radiation | | | | Granulocyte stimulating factor, Fibroblast | Ced 9 gene |
| Calcium | Heat shock | | | | growth factor, Transforming growth factor β1 | mutants in C- |
| Azide | | | | | Neurotrophic factor) | Elegans |
| Hydrogen peroxide | | | | | Hormones (Estrogen, Androgen, | Fas/Apo-1 |
| | | | | | Progesterone, ACTH) | (CD95) |
| | | | | | | IL-1β converting |
| | | | | | | enzyme |

Based on sequence homology, a large family of molecules collectively termed the nerve growth factor/TNF receptor family of apoptosis inducing signalling proteins has been identified. In addition, the extracellular domains of the TNF family bears significant homology to the open reading frames of several viruses. Over the past two years, ligands for most of the known receptors of the nerve growth factor/TNF receptor family have been identified. These ligands are all type II transmembrane proteins and show homology to TNF and lymphotoxin and therefore belong to a TNF family of molecules.

Tumor necrosis factor-α (herein referred to as “TNF”) is one of the prime signals which induces apoptosis in a wide range of cells. It was originally defined by its tumoricidal activity but is, in fact, a pleiotropic cytokine which elicits a wide spectrum of organismal and cellular responses such as cell proliferation, apoptosis, and inflammatory and immunoregulatory responses. The different cellular responses to TNF are signalled through cell surface receptors (p55 TNF-R1 and p75 TNF-R2), and their adaptor proteins, initiating distinct and separate signalling pathways. These separate signals can lead to opposing cellular effects as best exemplified by TNF's both apoptotic and anti-apoptotic role (Locksley et al., Cell 104, 487, 2001).

The strikingly different cellular responses to tumor necrosis factor, such as cell survival, activation and apoptosis, are signalled through the separate pathways. Discrete signalling is believed to be initiated by recruiting different types of adaptor proteins to the TNF receptor superfamily complexes. For example, the recruitment of a complex including FADD/MORT1 and in the case of some but not all receptors, TRADD, which leads to the further recruitment and activation of caspase 8 (and in human also caspase 10) leading to activation of so called “downstream” caspases and, subsequently, to cell death (Tartaglia et al., Cell 73, 213, 1993; Chinnaiyan et al., Science 274, 990, 1996). On the other hand, TNF induces the interaction of its receptor with a second class of adaptor proteins, TNFR-associated factors (TRAFs) to downstream signals, such as NF-κB-inducing kinase (NIK) to activate NF-κB (Locksley et al., 2001 supra; Arch et al., Genes & Devel. 12, 2821, 1998).

The cell membrane has two specialized receptors for TNF: TNF-R1 and TNF-R2. The binding of TNF to TNF-R1 has been shown to upregulate the pathway that leads to activating the caspases. Fas (also known as Apo-1 or CD95), is another receptor of extrinsic apoptotic signals in the cell membrane, and belongs to the TNF receptor superfamily. The Fas ligand is a transmembrane protein, and is part of the TNF family. The interaction between Fas and FasL results in the formation of the death-inducing signalling complex (DISC), which contains the Fas-associated death domain protein (FADD) and caspases 8 and 10. In some types of cells, processed caspase-8 directly activates other members of the caspase family and triggers the execution of apoptosis whereas in other types of cells, the Fas DISC induces a feed-back loop which spirals into increasing release of pro-apoptotic factors from mitochondria, and the amplified activation of caspase-8.

Downstream from TNF-R1 and Fas activation a balance between pro- (like BAX, BID, or BAD) and anti-apoptotic (Bcl-X1 and Bcl-2) members of the Bcl-2 family is compromised.

This balance is the proportion of pro-apoptotic homodimers that form in the outer-membrane of the mitochondrion. The homodimers (of molecules like BAK and BAX) are required in order to make the mitochondrial membrane permeable for the release of caspase activators. Just how BAX and BAK are controlled under the normal conditions of cells that are not undergoing apoptosis is incompletely understood, but it has been found that a mitochondrial outer-membrane protein, VDAC2, interacts with BAK to keep this potentially-lethal apoptotic effector under control. When the death signal is received, products of the activation cascade—such as tBID, BIM or BAD—displace VDAC2: BAK and BAX are activated, and the mitochondrial outer-membrane becomes permeable.

However, these pathways are subject to regulatory mechanisms. Accordingly there is not a simple and direct relationship between the reception of TNF or FasL and the complete execution of an apoptotic pathway. Fas, for instance, has been implicated in cell proliferation, as has TNF. Both Fas and TNF-R1 trigger events that activate the transcription factor nuclear factor kappa-B (NF-κB), which induces the expression of genes that play an important role in diverse biological processes, including cell growth, cell death, cell development, and immune responses.

The apoptotic pathway is therefore complex and its elucidation and analysis is constantly under examination.

BH3-only proteins (Bad, Bik/Blk/Nbk), Hrk/DP5, Bid, Bim/Bod, Bmf, Noxa and Puma/Bbc3) are evolutionarily conserved pro-apoptotic members of the Bcl-2 family that play a role in the initiation of developmentally programmed cell death and cytotoxic stress-induced apoptosis (Huang and Strasser, Cell 103:839-842 (2000)). They can be activated through a range of transcriptional or post-translational processes and they kill cells by a process that requires the Bax/Bak-like pro-apoptotic subgroup of the Bcl-2 family (Huang and Strasser, 2000, supra). Bid is an unusual BH3-only protein (Wang et al., Genes and Development 10:2859-2869, 1996) because it is activated by caspase-mediated proteolysis (Li et al., Cell 94:491-501, 1998; Luo et al., Cell 94:481-490, 1998). This cleavage promotes post-translational N-myristoylation of Bid, thereby facilitating its translocation to the mitochondrial outer membrane where it initiates apoptosis signalling (Zha et al., Science 290:1761-1765, 2000).

However, despite the general acceptance that BH3-only proteins exhibit pro-apoptotic activity, the reality in relation to the mechanisms regulating apoptosis across different cell types and in the context of differing physiological or immunological circumstances is such that the role of BH3-only proteins in this regard is significantly more complex than may have been initially postulated. For example, over-activation of the adaptive or innate immune system can lead to TNF-mediated fatal hepatocyte destruction. Although Bid, a pro-apoptotic BH3-only member of the Bcl-2 family that can be activated through caspase-mediated proteolysis, is essential for Fas ligand-induced liver injury, its loss has no impact on fatal hepatocyte destruction triggered by polyclonal T cell activation, this being mediated by membrane-anchored TNF. Moreover, Bid-deficiency affords only limited protection against injection with LPS plus the liver-specific transcriptional inhibitor galactosamine, a stimulus that kills hepatocytes through secreted TNF.

Accordingly, in terms of developing means of regulating cellular apoptosis in a targeted manner, there remains a need to elucidate the complex intracellular signalling mechanisms which function to result in the induction of apoptosis in the highly selective manner which is often observed to occur.

In work leading up to the present invention it has been determined that in the context of TNF-mediated hepatocyte apoptosis, downregulation of the functionality of Bim and/or Bid afforded significant protection against soluble TNF mediated apoptosis while the downregulation of the functionality of Bim afforded protection against membrane-bound TNF-mediated apoptosis. These findings, in their own right, confirm the complex nature of apoptosis regulation and, further, the role of BH3-only members within this regime.

The elucidation of the signalling mechanism which regulates TNF-mediated cellular apoptosis has now facilitated the development of methodology directed to modulating this type of cellular apoptosis by regulating the functioning of Bim and/or Bid. The method of the present invention is useful, inter alia, in the treatment and/or prophylaxis of conditions characterised by aberrant, unwanted or otherwise inappropriate TNF-mediated cellular apoptosis, in particular hepatocyte apoptosis, such as diseases characterised by severe hepatocellular destruction. Also facilitated has been the development of methods for screening for agents capable of modulating TNF-mediated cellular apoptosis.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method of modulating mammalian TNF-mediated cellular apoptosis, said method comprising modulating the functional level of Bim in an apoptotic or pre-apoptotic cell wherein upregulating said level facilitates the induction of TNF-mediated cellular apoptosis and downregulating said level inhibits or reduces TNF-mediated cellular apoptosis.

In another aspect there is provided a method of modulating mammalian TNF-mediated cellular apoptosis, which apoptosis is pathological cellular apoptosis, said method comprising modulating the functional level of Bim in an apoptotic or pre-apoptotic cell wherein upregulating said level facilitates the induction of TNF-mediated cellular apoptosis and downregulating said level inhibits or reduces TNF-mediated cellular apoptosis.

Yet another aspect of the present invention is directed to a method of modulating mammalian TNF-mediated hepatocyte apoptosis, said method comprising modulating the functional level of Bim in an apoptotic or pre-apoptotic hepatocyte wherein upregulating said level facilitates the induction of TNF-mediated hepatocyte apoptosis and downregulating said level inhibits or reduces TNF-mediated hepatocyte apoptosis.

In still another aspect the present invention provides a method of modulating mammalian TNF-mediated cellular apoptosis, which TNF is soluble, said method comprising modulating the functional level of Bim or Bid in an apoptotic or pre-apoptotic cell wherein upregulating said level facilitates the induction of TNF-mediated cellular apoptosis and downregulating said level inhibits or reduces TNF-mediated cellular apoptosis.

In still another aspect of the present invention there is provided a method of modulating mammalian TNF-mediated cellular apoptosis, which TNF is soluble, said method comprising modulating the functional level of Bim and Bid in an apoptotic or pre-apoptotic cell wherein upregulating said level facilitates the induction of TNF-mediated cellular apoptosis and downregulating said level inhibits or reduces TNF-mediated cellular apoptosis.

In still another aspect of the present invention there is provided a method of modulating mammalian TNF-mediated cellular apoptosis, which TNF is soluble, said method comprising modulating the functional level of Bid in an apoptotic or pre-apoptotic cell wherein upregulating said level facilitates the induction of TNF-mediated cellular apoptosis and downregulating said level inhibits or reduces TNF-mediated cellular apoptosis.

In yet still another aspect there is provided a method of modulating mammalian TNF-mediated cellular apoptosis, which TNF is membrane bound, said method comprising modulating the functional level of Bim in an apoptotic or pre-apoptotic cell wherein upregulating said level facilitates the induction of TNF-mediated cellular apoptosis and downregulating said level inhibits or reduces TNF-mediated cellular apoptosis.

Still yet another aspect of the present invention directed to a method of modulating TNF-mediated cellular apoptosis in a mammal, said method comprising modulating the functional level of Bim in an apoptotic or pre-apoptotic cell in said mammal wherein upregulating said level facilitates the induction of TNF-mediated cellular apoptosis and downregulating said level inhibits or reduces TNF-mediated cellular apoptosis.

A further aspect of the present invention is directed to a method of modulating TNF-mediated hepatocyte apoptosis in a mammal, said method comprising modulating the functional level of Bim in an apoptotic or pre-apoptotic hepatocyte in said mammal wherein upregulating said level facilitates the induction of TNF-mediated hepatocyte apoptosis and downregulating said level inhibits or reduces TNF-mediated hepatocyte apoptosis.

Another further aspect of the present invention contemplates a method for the treatment and/or prophylaxis of a condition characterised by aberrant TNF-mediated cellular apoptosis in a mammal, said method comprising modulating the functional level of Bim in an apoptotic or pre-apoptotic cell in said mammal wherein upregulating said level facilitates the induction of TNF-mediated cellular apoptosis and downregulating said level inhibits or reduces TNF-mediated cellular apoptosis.

Still another further aspect of the present invention provides a method for the treatment and/or prophylaxis of a condition characterised by aberrant TNF-mediated hepatocyte apoptosis in a mammal, said method comprising modulating the functional level of Bim in an apoptotic or pre-apoptotic hepatocyte in said mammal wherein upregulating said level facilitates the induction of TNF-mediated cellular apoptosis and downregulating said level inhibits or reduces TNF-mediated cellular apoptosis.

In yet another further aspect there is provided a method for the treatment and/or prophylaxis of a condition characterised by unwanted TNF-mediated cellular apoptosis, said method comprising administering to said mammal an effective amount of an agent for a time and under conditions sufficient to downregulate the functional level of Bim in an apoptotic or pre-apoptotic cell.

In yet still another further aspect there is provided a method for the treatment and/or prophylaxis of a condition characterised by unwanted TNF-mediated cellular apoptosis, said method comprising administering to said mammal an effective amount of an agent for a time and under conditions sufficient to downregulate the functional level of Bim and Bid in an apoptotic or pre-apoptotic cell.

In still another further aspect there is provided a method for the treatment and/or prophylaxis of a condition characterised by unwanted TNF-mediated cellular apoptosis, said method comprising administering to said mammal an effective amount of an agent for a time and under conditions sufficient to downregulate the functional level of Bid in an apoptotic or pre-apoptotic cell.

Still yet another further aspect of the present invention relates to the use of an agent capable of modulating the functional level of Bim in an apoptotic or pre-apoptotic cell in the manufacture of a medicament for the treatment and/or prophylaxis of a condition characterised by aberrant TNF-mediated cellular apoptosis in a mammal wherein upregulating said level facilitates the induction of TNF-mediated cellular apoptosis and downregulating said level inhibits or reduces TNF-mediated cellular apoptosis.

In another aspect there is provided the use of an agent capable of modulating the functional level of Bim in an apoptotic or pre-apoptotic hepatocyte in the manufacture of a medicament for the treatment and/or prophylaxis of a condition characterised by aberrant TNF-mediated hepatocyte apoptosis in a mammal wherein upregulating said level facilitates the induction of TNF-mediated cellular apoptosis and downregulating said level inhibits or reduces TNF-mediated cellular apoptosis.

In yet another aspect there is provided the use of an agent capable of downregulating the functional level of Bim in an apoptotic or pre-apoptotic cell in the manufacture of a medicament for the treatment and/or prophylaxis of a condition characterised by unwanted TNF-mediated cellular apoptosis.

In still another aspect there is provided the use of an agent capable of downregulating the functional level of Bim and Bid in an apoptotic or pre-apoptotic cell in the manufacture of a medicament for the treatment and/or prophylaxis of a condition characterised by unwanted TNF-mediated cellular apoptosis.

In still another aspect there is provided the use of an agent capable of downregulating the functional level of Bid in an apoptotic or pre-apoptotic cell in the manufacture of a medicament for the treatment and/or prophylaxis of a condition characterised by unwanted TNF-mediated cellular apoptosis.

In yet still another aspect, the present invention contemplates a pharmaceutical composition comprising the modulatory agent as hereinbefore defined and one or more pharmaceutically acceptable carriers and/or diluents. Said agents are referred to as the active ingredients

Yet another aspect of the present invention relates to the agent as hereinbefore defined, when used in the method of the invention.

Another aspect of the present invention provides a method for detecting an agent capable of modulating TNF-mediated cellular apoptosis by modulating Bim or Bid functionality said method comprising contacting a cell or extract thereof containing Bim or Bid or its functional equivalent or derivative with a putative agent and detecting an altered expression phenotype.

In still another aspect the present invention provides a method for detecting an agent capable of modulating TNF-mediated cellular apoptosis by modulating Bim or Bid functionality said method comprising contacting a cell containing said Bim or Bid or its functional equivalent or derivative with a putative agent and detecting an altered apoptosis profile.

### BRIEF DESCRIPTION OF THE DRAWINGS

FIG. 1 illustrates that Bid-deficient mice are normally sensitive to ConA-induced hepatitis. (A) ConA induced fatal hepatitis is mediated by TNF but does not require Bid. Mice of the indicated genotypes were injected i.v. with either PBS or a lethal dose (30 μg/g body weight) of ConA. The mice were sacrificed after 6 hr, bled and their sera analysed for the liver enzyme ALT. Note that AST-levels could not reliably be measured in this experiment due to extensive hemolysis caused by ConA. (B) H&E stained histological liver sections from mice treated as in (A) (bars=50 μm). Pictures shown are representative of the analysis of at least 3 mice for each treatment and genotype.

FIG. 2 illustrates that Bid plays a limited role in LPS plus GalN induced hepatitis. (A) Bid-deficient mice and wt littermate controls were injected i.p with 10 ng of LPS in the presence of the liver-specific transcriptional inhibitor GalN (20 mg/mouse). Mice were sacrificed after 6 hr, bled and sera analysed for the liver enzymes ALT and AST. (B) Bid-deficient mice and wt littermate controls were injected i.p. with 10, 100 or 1000 ng LPS, all in the presence of 20 mg GalN, and analysed after 6 hr as in (A). Error bars represent standard deviation. (C) Histological examination of H&E stained liver sections of bid and wt mice subjected to the treatments indicated (bars=50 μm). Pictures shown are representative of the analysis of at least 3 mice for each treatment and genotype.

FIG. 3 is a graphical representation illustrating that caspase-8 is essential for ConA as well as LPS plus GalN-induced hepatitis. Mice lacking caspase-8 in hepatocytes (albumin Cre transgenic caspase-8-flox/flox) and littermate controls (albumin Cre transgenic caspase-8-flox/wt mice) were injected with either 30 μg ConA (A) or 100 ng LPS plus 20 mg GalN. At the time when the wt mice were moribund (8 hr), all animals were sacrificed and serum levels of ALT and AST measured. Data shown represent means+/−SD of 3-5 mice for each genotype and each treatment.

FIG. 4 is a graphical representation illustrating that the combined loss of Bid and Bim protects mice against LPS plus GalN-induced hepatitis and the loss of Bim protects against ConA-induced hepatocyte destruction. Double knock-out mice lacking both Bid and Bim and control animals (wt, bid^{-/-} or bim^{-/-}) were injected with either 30 μg ConA (A) or 100 ng LPS plus 20 mg GalN. At the time when the wt mice were moribund (8 hr), all animals were sacrificed and serum levels of ALT and AST measured. Data shown represent means+/−SD of 3-5 mice for each genotype and each treatment.

FIG. 5 is a schematic representation of a proposed model for TNF-mediated hepatocyte destruction.

FIG. 6 illustrates that Bid-deficient mice are resistant to Fas ligand-induced hepatitis. (A) Bid-deficient mice and wt littermate controls were injected i.v. with either PBS or a lethal dose (0.25 μg/g body weight) of crosslinked recombinant FasL (soluble human FasL with a FLAG epitope plus anti-FLAG antibody). Mice were sacrificed and analysed as in (A) after 80 min. (bid^{-/-} vs wt: p=0.006 for ALT, p=0.012 for AST) (B) Histological examination of liver sections from wt or bid^{-/-} mice injected 80 or 200 min earlier with FasL or carrier (PBS), respectively (bars=50 μm). Pictures shown are representative of the analysis of at least 5 mice for each treatment and genotype.

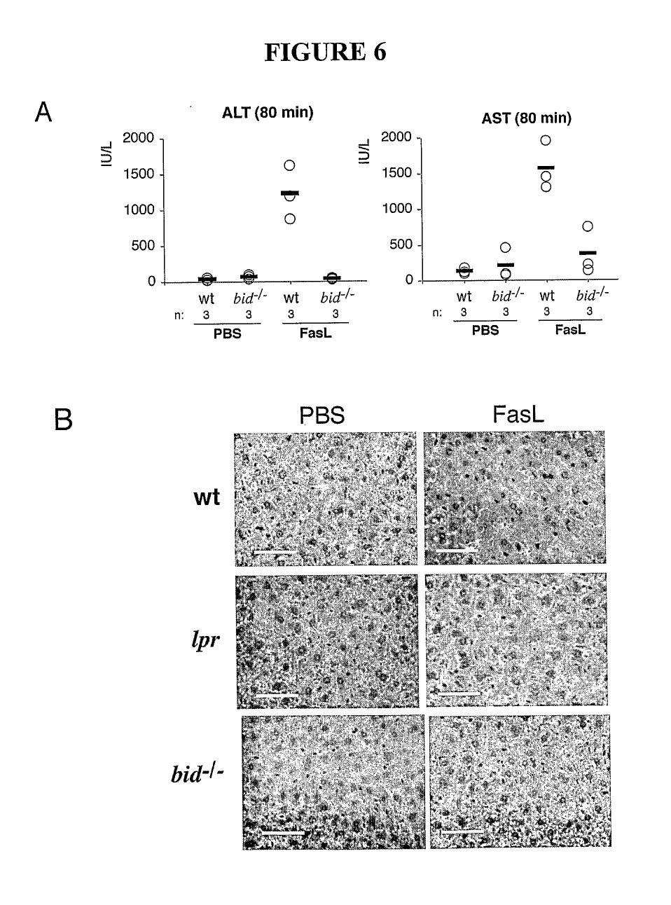

FIG. 7 is an image of caspase-8 activity, reflected by cleavage of Bid and caspase-7, in the liver of mice injected with anti-Fas Antibody or LPS plus GalN. Mice (wt) were injected with either anti-Fas antibody (Jo2 at 0.25 μg/g body weight) (A) or LPS (100 ng) plus GalN (20 mg) (B) and sacrificed after the indicated time points. Bid and caspase-7 processing were examined by probing Western blots with a rat anti-Bid monoclonal antibody (clone 2D1; TK, David C S Huang and AS, submitted) or with a mouse anti-caspase-7 monoclonal antibody (gift from Y. Lazebnik), respectively. Probing with a monoclonal antibody to β-actin served as a loading control, ‘C’ indicates an apoptotic cell-derived control lysate (growth factor-dependent FDC-P1 cells deprived of IL-3 for 36 hours).

FIG. 8 is an image illustrating the lack of evidence for Bim proteolysis in the liver of mice injected with LPS plus GalN. Mice (wt) were injected with LPS (100 ng) plus GalN (20 mg) and sacrificed after 0, 3 or 4 hr. Bim levels and possible post-translational modifications (e.g. proteolytic cleavage) were investigated by probing Western blots with a rabbit polyclonal antibody to Bim (Stressgen) or, as a loading control, with a monoclonal antibody to β-actin.

FIG. 9 is an image depicting that treatment with LPS+GalN or with ConA causes a hyperphosphorylation in Bim that does not require Caspase-8 or other caspases. Mice (wt) were injected with LPS (100 ng) plus GalN (20 mg) (a) or ConA (30 μg/g body weight) (b) and sacrificed at the time points indicated. Bim levels and possible post-translational modifications (e.g. mobility shift or proteolytic cleavage) were investigated by probing Western blots with a rabbit polyclonal antibody to Bim (Stressgen) or, as a loading control, with a monoclonal antibody to β-actin. (c) Mice (wt) were injected with LPS (100 ng) plus GalN (20 mg) or ConA (30 μg/g body weight) and sacrificed after 4 hours. Total protein extracts were prepared from the liver in the absence of phosphatase inhibitors and left untreated or treated in vitro with λ-phosphatase prior to analysing Bim modifications by Western blotting. (d) Mice (wt), with or without pretreatment with 20 mg/kg of the pan-caspase inhibitor Q-VD-oph, were injected with LPS (100 ng) plus GalN (20 mg) or ConA (30 μg/g body weight) and sacrificed 4 hr later. Total protein extracts from the livers of these animals were probed by Western blotting for Bim. (e) Mice lacking caspase-8 in hepatocytes (C8: albumin Cre transgenic caspase-8-flox/flox) and littermate controls (wt: albumin Cre transgenic caspase-8-flox/wt) were injected with 100 ng LPS plus 20 mg GalN or with 30 μg/g body weight ConA and sacrificed after 4 hr. Total protein extracts derived from the livers of these animals were probed by Western blotting for Bim and β-actin (loading control).

### DETAILED DESCRIPTION OF THE INVENTION

The present invention is predicated, in part, on the elucidation of the role of BH3-only proteins in the context of TNF-mediated cellular apoptosis. In particular, it has been determined that this particular class of cellular apoptosis is mediated by Bim, alone, in the context of membrane-bound TNF signalling and by Bim and Bid in the context of soluble TNF signalling. These determinations have now permitted the rational design of therapeutic and/or prophylactic methods for treating conditions characterised by aberrant or unwanted TNF-mediated cellular apoptosis, in particular unwanted TNF-mediated hepatocyte apoptosis. Further, there is facilitated means for screening for agents which are useful for modulating the functional levels of Bim and/or Bid in the context of regulating TNF-mediated cellular apoptosis.

Accordingly, one aspect of the present invention is directed to a method of modulating mammalian TNF-mediated cellular apoptosis, said method comprising modulating the functional level of Bim in an apoptotic or pre-apoptotic cell wherein upregulating said level facilitates the induction of TNF-mediated cellular apoptosis and downregulating said level inhibits or reduces TNF-mediated cellular apoptosis.

Reference to “apoptosis” should be understood as a reference to the controlled intracellular process which is characterised by chromatin condensation and cell shrinkage in the early stages followed by nuclear and cytoplasmic fragmentation. Without limiting the present invention to any one theory or mode of action, this leads to the formation of apoptotic bodies which can be phagocytosed. A cascade of caspase activity is invoked, leading both to the cleavage of procaspases to generate further active caspases and the cleavage of DNA. It is this DNA cleavage event which results in the characteristic “laddering pattern” which is seen on gels. To this end, it would be understood that the method of the present invention is directed to modulating the level of Bim in an apoptotic cell or a pre-apoptotic cell. Reference to an “apoptotic cell” should be understood as a reference to any cell in which the apoptosis process has commenced or any cell which has received the apoptosis signal but which may not yet have commenced the intracellular cascade of steps which are characteristic of apoptosis. Reference to a “pre-apoptotic” cell should be understood as a reference to a cell which has not yet received an apoptotic signal. Since the commitment of a cell to the apoptosis process via a TNF-mediated mechanism occurs via the binding of TNF to a cell surface receptor, such as but not limited to TNF-R1 or TNF-R2, it should further be understood that reference to a pre-apoptotic cell is reference to a cell which is capable of undergoing TNF-mediated apoptosis. Without limiting the present invention to any one theory or mode of action, there exists evidence that the early stages of the apoptosis process are reversible. Accordingly, the method of the present invention can be applied to either prevent the onset of apoptosis events in a tissue which may be predisposed to this occurring, such as in a diseased liver, or it may be directed to downregulating or reversing an apoptosis process which has commenced.

As detailed hereinbefore, the regulation of apoptotic processes can occur both in the context of the normal development and functioning of an organism (herein referred to as “normal apoptosis”) or may be associated with an unwanted pathology (herein referred to as “pathological apoptosis”). For example, pathological apoptosis may occur in the context of autoimmune conditions or other disease states which lead to unwanted tissue destruction which is characterised by unwanted apoptosis events, or neoplastic conditions which are characterised by the absence of the apoptotic events which would be required to prevent the onset of the neoplastic state. Preferably, the subject apoptosis is pathological apoptosis.

There is therefore preferably provided a method of modulating mammalian TNF-mediated cellular apoptosis, which apoptosis is pathological cellular apoptosis, said method comprising modulating the functional level of Bim in an apoptotic or pre-apoptotic cell wherein upregulating said level facilitates the induction of TNF-mediated cellular apoptosis and downregulating said level inhibits or reduces TNF-mediated cellular apoptosis. Reference to “cellular” apoptosis should be understood as reference to the apoptosis of any cell which is located either in vitro or in vivo. To this end, reference to “cell” should be understood as a reference to any normal or abnormal cell located either in vitro or in vivo. The cell may be one which has been genetically manipulated or it may exist in its original form. A cell which is the subject of treatment in vitro may have been freshly isolated from an individual (such as an individual who may be the subject of treatment) or it may have been sourced from a non-fresh source, such as from a culture (for example, where cell numbers were expanded and/or the cells were cultured so as to render them receptive to differentiative signals) or a frozen stock of cells (for example, an established cell line), which had been isolated at some earlier time point either from an individual or from another source. It should also be understood that the subject cells, prior to undergoing treatment, may have undergone some other form of treatment or manipulation, such as but not limited to enrichment or purification, modification of cell cycle status or the formation of a cell line. Accordingly, the subject cell may be a primary cell or a secondary cell. A primary cell is one which has been isolated from an individual. A secondary cell is one which, following its isolation, has undergone some form of in vitro manipulation such as the preparation of a cell line, prior to the application of the method of the invention. Preferably, the subject cell is a cell susceptible to TNF-mediated apoptosis. More preferably, the subject cell is a hepatocyte, macrophage or fibroblast. Even more preferably, the subject cell is a hepatocyte.

Accordingly, the present invention is more particularly directed to a method of modulating mammalian TNF-mediated hepatocyte apoptosis, said method comprising modulating the functional level of Bim in an apoptotic or pre-apoptotic hepatocyte wherein upregulating said level facilitates the induction of TNF-mediated hepatocyte apoptosis and downregulating said level inhibits or reduces TNF-mediated hepatocyte apoptosis.

Preferably, said hepatocyte apoptosis is pathological hepatocyte apoptosis.

By “TNF-mediated” is meant that the subject apoptosis occurs, either directly or indirectly, by the actions of TNF. Without limiting the present invention to any one theory or mode of action TNF is a 157 amino acid intracellular signalling cytokine which is produced mainly by macrophages. It is thought to be one of the major extrinsic mediators of apoptosis. TNF can function to induce apoptosis in cells in the context of either its membrane bound form or in its soluble form. For example, hepatocyte apoptosis which results from the systemic activation of T cells is linked to the actions of a membrane-bound form of the TNF which provides the apoptotic signal. This is exemplified herein in the context of concanavalin A activation of T cells which leads to fatal hepatitis due to the hepatocyte apoptosis induction which is mediated by membrane bound TNF. Conversely, the administration of LPS and D-(+)-galactosamine results in hepatocyte apoptosis via the actions of soluble TNF.

Reference to “TNF” should be understood as a reference to all forms of TNF, including for example TNF-α and TNF-β and functional derivatives, homologues, orthologues, analogues, chemical equivalents and mimetics thereof. It would be appreciated that reference to derivatives, and the like of TNF is likely to be relevant in the context of individuals who are receiving, as a therapeutic or prophylactic treatment, exogenously administered molecules which exhibit TNF functionality. Such molecules may take the form of active fragments of TNF, homologous or orthologous forms of TNF or chemical/synthetic mimetics or analogues of TNF. To this end, reference to “TNF” should also be understood to include reference to any isoforms which arise from alternative splicing of TNF mRNA or mutants or polymorphic variants of TNF. It should also be understood to include reference to any other molecule which exhibits TNF functional activity to the extent that the subject molecule mimics one or more TNF signalling events by inducing signalling through a TNF or TNF-like receptor. Since the method of the present invention is directed to modulating cellular apoptosis by modulating an intracellular signalling event which has been induced as a result of the interaction of TNF with its receptor, this methodology can be applied to modulating such an outcome, irrespective of whether it has been induced by the interaction of TNF with a TNF receptor or the interaction of a TNF mimetic, such as a naturally occurring or non-naturally occurring mimetic or analogue, with the subject receptor. It is conceivable, for example, that there may exist naturally or non-naturally occurring TNF mimetics (for example, toxins or drugs) which, if they were introduced into an individual, would induce unwanted TNF-like cellular apoptosis due to their interaction with the TNF receptor. Accordingly, the present invention should be understood to extend to the modulation of such cellular apoptosis which is herein defined as falling within the scope of being “TNF-mediated”.

Preferably, said TNF is TNF-α or TNF-β.

More preferably, said TNF is TNF-α.

In terms of the present invention, it has been determined that in the context of TNF-mediated cellular apoptosis, in particular hepatocyte apoptosis, the apoptotic process is mediated by differing intracellular pathways depending on whether the TNF signal is provided by a membrane bound form of TNF or a soluble form of TNF. In the context of membrane bound TNF, it has been determined that reduction in the level of Bim is effective to downregulate cellular apoptosis. In the context of the apoptotic signal provided by soluble TNF, however, although downregulation in Bim levels is effective to reduce the extent of cellular apoptosis, to most effectively achieve the prevention of apoptosis a reduction in the levels of both Bim and Bid is desirable. This contrasts to the situation with membrane-bound TNF mediated apoptosis which does not appear to be dependent on Bid signalling. Still further, in the context of the apoptotic signal provided by soluble TNF, it has also been determined that reduction in the level of Bid, alone, is effective to reduce cellular apoptosis. However, analogous to the situation with Bim, although downregulation of Bid alone will reduce the extent of cellular apoptosis induced by soluble TNF, to most extensively reduce soluble TNF medicated apoptosis both Bim and Bid should be reduced. The identification of these pathways has now provided a highly sensitive and sophisticated means of regulating TNF-mediated apoptosis.

Accordingly, in one embodiment of the present invention there is provided a method of modulating mammalian TNF-mediated cellular apoptosis, which TNF is soluble, said method comprising modulating the functional level of Bim or Bid in an apoptotic or pre-apoptotic cell wherein upregulating said level facilitates the induction of TNF-mediated cellular apoptosis and downregulating said level inhibits or reduces TNF-mediated cellular apoptosis.

In another preferred embodiment there is provided a method of modulating mammalian TNF-mediated cellular apoptosis, which TNF is soluble, said method comprising modulating the functional level of Bim and Bid in an apoptotic or pre-apoptotic cell wherein upregulating said level facilitates the induction of TNF-mediated cellular apoptosis and downregulating said level inhibits or reduces TNF-mediated cellular apoptosis.

Preferably, said cellular apoptosis is apoptosis of a cell susceptible to TNF-mediated apoptosis. More preferably said cellular apoptosis is hepatocyte, macrophage or fibroblast apoptosis. Even more preferably said cellular apoptosis is hepatocyte apoptosis and most preferably pathological hepatocyte apoptosis.

In another preferred embodiment there is provided a method of modulating mammalian TNF-mediated cellular apoptosis, which TNF is membrane bound, said method comprising modulating the functional level of Bim in an apoptotic or pre-apoptotic cell wherein upregulating said level facilitates the induction of TNF-mediated cellular apoptosis and downregulating said level inhibits or reduces TNF-mediated cellular apoptosis.

Preferably, said cellular apoptosis is apoptosis of a cell susceptible to TNF-mediated apoptosis. More preferably said cellular apoptosis is hepatocyte, macrophage or fibroblast apoptosis. Even more preferably said cellular apoptosis is hepatocyte apoptosis and most preferably pathological hepatocyte apoptosis.

It would be appreciated by the person of skill in the art that although the downregulation of Bid has little functional impact in terms of regulating the apoptosis mediated by membrane-bound TNF, it may nevertheless be clinically convenient to modulate levels of both Bid and Bim as part of an overall treatment regime, particularly in the situation where unwanted apoptosis is occurring via the actions of both membrane bound and soluble TNF. Conversely, in some situations it may be clinically desirable to pursue only the modulation of Bim levels. Where this occurs, it would be appreciated by the person of skill in the art that even if soluble TNF is involved in the apoptosis events in issue, the downregulation of Bim alone may nevertheless provide sufficient therapeutic benefit such that even if not all the unwanted apoptotic events have been inhibited, the outcome for the patient in issue is nevertheless acceptable.

Reference to “Bim” and “Bid” should be understood as a reference to all forms of these molecules and to fragments, mutants or variants thereof. It should also be understood to include reference to any isoforms which may arise from alternative splicing of Bim or Bid mRNA or mutant or polymorphic forms of Bim or Bid. For example, there are at least three known isoforms of human and mouse Bim, these being Bim_{S}, Bim_{L} and Bim_{EL}. Without limiting the present invention to any one theory or mode of action, Bim and Bid are known as a “BH3-only” proteins since the only Bcl-2 homology region which they encompass is BH3. They thereby form a novel Bcl-2 related BH3-only pro-apoptotic group which also comprises, for example, Bik/Nbk and Hrk. The BH3-only proteins share with each other and the Bcl-2 family at large only the 9-16 amino acid BH3 region and they are essential for initiation of apoptosis signalling (Huang, 2000). BH3-only proteins are regulated by a range of transcriptional and post-translational mechanisms (Puthalakath et al. Cell Death Differ. 2002, 9(5):505-12) and experiments with gene knockout mice have shown that different members of this subgroup are required for the execution of different death stimuli (Huang, 2000).

Reference to “modulating” should be understood as a reference to upregulating or downregulating the subject apoptosis. Reference to “downregulating” apoptosis should therefore be understood as a reference to preventing, reducing (e.g. slowing) or otherwise inhibiting one or more aspects of this event while reference to “upregulating” should be understood to have the converse meaning.

Reference to the “functional level” of Bim and Bid should be understood as a reference to the capacity of these protein molecules to perform their normal range of activities. To this end, changes to the functional level of Bim and Bid can be caused in at least two ways, as follows:
(i) changes to the concentration of Bim or Bid expression product may occur. For example intracellular concentrations of Bim or Bid may be either reduced or increased, for example by modulating promoter activity, which thereby alters levels of expression; or
(ii) changes to the activity of Bim or Bid. This may or may not also involve changes to the concentration of Bim or Bid. The activity of these molecules may be modulated by any one of a number of mechanisms including blocking Bim or Bid binding sites, for example using antibodies, or using small molecule competitive inhibitors to prevent binding of these molecules to their interacting partners.

To this end, such modulation may be achieved by any suitable means and includes:
(i) Modulating absolute levels of the proteinaceous form of Bim and Bid (for example increasing or decreasing intracellular Bim and Bid concentrations) such that either more or less Bim and Bid are available for activation and/or to interact with their downstream targets.
(ii) Agonising or antagonising Bim and Bid such that the functional effectiveness of any given Bim or Bid molecule is either increased or decreased. For example, increasing the half life of Bim and Bid may achieve an increase in the overall level of Bim and Bid activity without actually necessitating an increase in the absolute intracellular concentration of Bim and Bid. Similarly, the partial antagonism of Bim and Bid, for example by coupling Bim and Bid to a molecule that introduces some steric hindrance in relation to the binding of Bim and Bid to their downstream targets, may act to reduce, although not necessarily eliminate, the effectiveness of Bim and Bid signalling. Accordingly, this may provide a means of down-regulating Bim and Bid functioning without necessarily downregulating absolute concentrations of Bim and Bid.

In terms of achieving the up- or downregulation of Bim and Bid functioning, means for achieving this objective would be well known to the person of skill in the art and include, but are not limited to:
(i) Introducing into a cell a nucleic acid molecule encoding Bim or Bid or functional equivalent, derivative or analogue thereof in order to upregulate the capacity of said cell to express Bim or Bid.
(ii) Introducing into a cell a proteinaceous or non-proteinaceous molecule which modulates transcriptional and/or translational regulation of a Bim or Bid gene.
(iii) Introducing into a cell the Bim or Bid expression product or a functional derivative, homologue, analogue, equivalent or mimetic thereof.
(iv) Introducing a proteinaceous or non-proteinaceous molecule which functions as an antagonist to the Bim or Bid expression product.
(v) Introducing a proteinaceous or non-proteinaceous molecule which functions as an agonist of the Bim or Bid expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the Bim and Bid expression products or small molecules capable of acting as agonists or antagonists. Agonists may be any compound capable of activating Bim or Bid or otherwise increasing the normal biological function of Bim or Bid. In terms of Bid, such agonists include proteases capable of activating Bid such as caspases, for example caspase 8 and 10, granzymes, cathepsins and calpains. Without limiting the present invention to any one theory or mode of action, caspases activate Bid by proteolytic processing to tBid. In respect of Bim, such agonists include kinases and phosphatases that interfere with sequestration of Bim to the microtubule-associated dynein motor complex; those kinases and phosphatases which prevent the degradation of Bim) for example, by the ubiquin/proteasome pathway) and which promote Bim translocation to mitochondria (for example JNK kinase and protein phosphatase 2A). Other examples of agonists include those agents which act to stimulate the interaction of Bid and Bim with other bcl-2 family members and mimetics such as BH3 mimetic compounds. Agonists also include those molecules which enhance transcriptional and/or translational regulation of a Bim or Bid gene, for example FOXO transcription factors. Chemical agonists may not necessarily be derived from the Bim and Bid expression products but may share certain conformational similarities. Alternatively, chemical agonists may be specifically designed to meet certain physiochemical properties.

Antagonists may be any compound capable of blocking, inhibiting or otherwise preventing Bim and Bid from carrying out their normal biological function. Such antagonists include inhibitors of caspase. granzyme, cathepsin and calpain, which act to prevent the activation of Bid or Bim and kinases and phosphatases such as ERK 1/2 which act to block Bim activation. With respect to Bim, such antagonists also include kinases and phosphatases that promote sequestration of Bim to the microtubule-associated dynein motor complex, those which promote degradation of Bim (for example, by the ubiquitin/proteasome pathway) such as ERK kinase, and those which prevent translocation of Bim to mitochondria. Antagonists also include chemical agents which act to interfere with the interaction of Bim with other bcl-2 family members. Still further, antagonists include monoclonal antibodies and antisense nucleic acids which prevent transcription or translation of Bim and Bid genes or mRNA in mammalian cells. Modulation of expression may also be achieved utilising antigens, RNA, ribozymes, DNAzymes, RNA aptamers, antibodies or molecules suitable for use in cosuppression. The proteinaceous and non-proteinaceous molecules referred to in points (i)-(v), above, are herein collectively referred to as “modulatory agents”.

The proteinaceous and non-proteinaceous molecules referred to in points (i)-(v) above, are herein collectively referred to as “modulatory agents”.

Screening for the modulatory agents hereinbefore defined can be achieved by any one of several suitable methods including, but in no way limited to, contacting a cell comprising the Bim and/or Bid genes or functional equivalent or derivative thereof with an agent and screening for the modulation of Bim or Bid protein production or functional activity, modulation of the expression of a nucleic acid molecule encoding Bim or Bid or modulation of the activity or expression of a downstream Bim or Bid cellular target. Detecting such modulation can be achieved utilising techniques such as Western blotting, electrophoretic mobility shift assays and/or the readout of reporters of Bim and Bid activity such as luciferases, CAT and the like.

It should be understood that the Bim or Bid genes or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate Bim or Bid activity, at either the nucleic acid or expression product levels, or the gene may require activation—thereby providing a model useful for, inter alia, screening for agents which up regulate Bim or Bid expression. Further, to the extent that a Bim or Bid nucleic acid molecule is transfected into a cell, that molecule may comprise the entire Bim or Bid gene or it may merely comprise a portion of the gene such as the portion which regulates expression of the Bim or Bid product. For example, the Bim or Bid promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively. One might also measure Bim or Bid activation directly.

The invention also provides methods for identifying/screening for modulators (e.g., inhibitors, activators) of Bim or Bid, using arrays. Potential modulators, including small molecules, nucleic acids, polypeptides (including antibodies) can be immobilized to arrays. Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of Bim or Bid. One or more, or, all the transcripts of a cell can be measured by hybridization of a sample comprising transcripts from the cell, or, nucleic acids representative of or complementary to transcripts of a cell, by hybridization to immobilized nucleic acids on an array, or “biochip.” By using an “array” of nucleic acids on a microchip, some or all of the transcripts from a cell can be simultaneously quantified. Polypeptide arrays can be used to simultaneously quantify a plurality of proteins. Small molecule arrays can be used to simultaneously analyze a plurality of binding activities.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer et al. (1997) Biotechniques 23:1087-1092; Kern (1997) Biotechniques 23:120-124; Solinas-Toldo et al. (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface.

Other identification methods include the yeast two-hybrid system in which full-length Bim or Bid polypeptide or fragments are expressed in yeast as “bait” fusion proteins in a screen against a cDNA library of “prey” fusion proteins. The fusion components of the screening system are typically the transactivation domain and DNA binding domain of a transcription factor such as yeast GAL4. When bait and prey bind each other, GAL4 transcriptional activation activity is reconstituted, upregulating transcription of a reporter gene construct. Such reporter constructs can be composed of GAL4 DNA binding sites upstream of a minimal promoter and marker gene such as lacZ, and library clones with increased reporter gene activity are identified by staining with β-D-galactoside.

In another example, the subject of detection could be a downstream Bim or Bid regulatory target, rather than Bim or Bid itself. For example, modulation of Bim or Bid activity can be detected by screening for the modulation of the functional activity of a hepatocyte. This is an example of an indirect system where modulation of Bim or Bid expression, per se, is not the subject of detection. Rather, modulation of the molecules and mechanisms which regulate the function or expression of Bim or Bid.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the Bim or Bid nucleic acid molecule or expression product itself. Accordingly, these methods provide a mechanism of detecting agents which modulate Bim or Bid expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region and/or its entry to a cell.

The term “expression” refers to the transcription and translation of a nucleic acid molecule. Reference to “expression product” is a reference to the product produced from the transcription and translation of a nucleic acid molecule. Reference to “modulation” should be understood as a reference to upregulation or downregulation.

“Derivatives” of the molecules herein described (for example Bim or Bid or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, Bim or Bid or derivative thereof may be fused to a molecule to facilitate its entry into a cell or its directed delivery to a tissue, such as the livers. Analogs of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, Si RNAs, PCR primers, antisense molecules, molecules suitable for use in co-suppression and fusion of nucleic acid molecules. Derivatives of nucleic acid sequences also include degenerate variants.

A “variant” or “mutant” of Bim or Bid should be understood to mean molecules which exhibit at least some of the functional activity of the form of Bim or Bid of which it is a variant or mutant. A variation or mutation may take any form and may be naturally or non-naturally occurring.

An “orthologue” is meant that the molecule is derived from a species other than that which is being treated in accordance with the method of the present invention. This may occur, for example, where it is determined that a species other than that which is being treated produces a form of Bim or Bid which exhibits similar and suitable (or even enhanced) functional characteristics to that of the Bim or Bid which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes, such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (e.g., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing Bim or Bid analogues which exhibit properties such as more potent pharmacological effects. Bim or Bid or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of Bim or Bid, it may also be desirable to identify and utilise molecules which function agonistically or antagonistically to Bim or Bid in order to up- or downregulate the functional activity of Bim or Bid in relation to modulating apoptosis. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of Bim or Bid capable of acting as agonists or antagonists. Chemical agonists may not necessarily be derived from Bim or Bid but may share certain conformational similarities. Alternatively, chemical agonists may be specifically designed to mimic certain physiochemical properties of Bim or Bid. Antagonists may be any compound capable of blocking, inhibiting or otherwise preventing Bim or Bid from carrying out its normal biological functions. Antagonists include monoclonal antibodies specific for Bim or Bid or parts of Bim or Bid.

Analogues of Bim or Bid or of Bim or Bid agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The method of the present invention contemplates the modulation of apoptosis both in vitro and in vivo. Although the preferred method is to treat an individual in vivo it should nevertheless be understood that it may be desirable that the method of the invention may be applied in an in vitro environment, for example to provide an in vitro model of apoptosis. In another example the application of the method of the present invention in an in vitro environment may extend to providing a readout mechanism for screening technologies such as those hereinbefore described. That is, molecules identified utilising these screening techniques can be assayed to observe the extent and/or nature of their functional effect on apoptosis in accordance with the method of the present invention.

Although the preferred method is to downregulate apoptosis (for example in order to treat diseases characterised by unwanted hepatocellular destruction), it should be understood that there may also be circumstances in which it is desirable to upregulate apoptosis, such as in the context of treating a hepatic tumor.

Accordingly, another aspect of the present invention directed to a method of modulating TNF-mediated cellular apoptosis in a mammal, said method comprising modulating the functional level of Bim in an apoptotic or pre-apoptotic cell in said mammal wherein upregulating said level facilitates the induction of TNF-mediated cellular apoptosis and downregulating said level inhibits or reduces TNF-mediated cellular apoptosis.

Preferably, said cellular apoptosis is apoptosis of a cell susceptible to TNF-mediated apoptosis. More preferably said cellular apoptosis is hepatocyte, macrophage or fibroblast apoptosis.

More particularly, the present invention is directed to a method of modulating TNF-mediated hepatocyte apoptosis in a mammal, said method comprising modulating the functional level of Bim in an apoptotic or pre-apoptotic hepatocyte in said mammal wherein upregulating said level facilitates the induction of TNF-mediated hepatocyte apoptosis and downregulating said level inhibits or reduces TNF-mediated hepatocyte apoptosis.

Preferably, said hepatocyte apoptosis is pathological hepatocyte apoptosis.

More preferably, said TNF is TNF-α or TNF-β.

Most preferably, said TNF is TNF-α.

Still more preferably, said TNF-α is soluble TNF-α and said method comprises modulating the functional level of Bim and/or Bid and yet more preferably Bim and Bid.

In another preferred embodiment, said TNF-α is membrane bound TNF-α and said method comprises modulating Bim alone.

A further aspect of the present invention relates to the use of the invention in relation to the treatment and/or prophylaxis of disease conditions or other unwanted conditions. Without limiting the present invention to any one theory or mode of action, the regulation of apoptosis is an essential requirement in terms of controlling cellular populations, for example terms of eliminating defective or unnecessary populations of cells. However, in some disease states the signals which control apoptotic processes become defective. This can be evidenced in terms of cellular populations which proliferate in an uncontrolled manner and lead to tumour formation or cellular populations which are destroyed as part of the progression of certain pathologies.

The present invention therefore contemplates a method for the treatment and/or prophylaxis of a condition characterised by aberrant TNF-mediated cellular apoptosis in a mammal, said method comprising modulating the functional level of Bim in an apoptotic or pre-apoptotic cell in said mammal wherein upregulating said level facilitates the induction of TNF-mediated cellular apoptosis and downregulating said level inhibits or reduces TNF-mediated cellular apoptosis.

Preferably, said cellular apoptosis is apoptosis of a cell susceptible to TNF-mediated apoptosis. More preferably said cellular apoptosis is hepatocyte, macrophage or fibroblast apoptosis. Even more preferably said cellular apoptosis is hepatocyte apoptosis and most preferably pathological hepatocyte apoptosis.

There is therefore more preferably provided a method for the treatment and/or prophylaxis of a condition characterised by aberrant TNF-mediated pathological hepatocyte apoptosis in a mammal, said method comprising modulating the functional level of Bim in an apoptotic or pre-apoptotic hepatocyte in said mammal wherein upregulating said level facilitates the induction of TNF-mediated cellular apoptosis and downregulating said level inhibits or reduces TNF-mediated cellular apoptosis.

Reference to “aberrant” apoptosis should be understood as a reference to either the absence of apoptosis where it would be required to remove a defective or otherwise unwanted population of cells or to the situation where the apoptosis which is characteristic of the condition in issue is unwanted in that, for example, it contributes to extensive tissue damage. Examples of conditions treatable in accordance with the method of the invention include, but are not limited to those associated with unwanted membrane bound TNF-mediated cellular apoptosis, such as autoimmune disease, graft-vs-host disease, transplant rejection and viral infections (eg. viral hepatitis); and conditions associated with unwanted soluble TNF-mediated cellular apoptosis, such as inflammation, sepsis and septic shock. Further examples of conditions treatable in accordance with the method of the invention include, but are not limited to conditions which result in the destruction of hepatic tissue, for example Hepatitis, alcoholic liver disease, conditions caused by drug overdoses and hepatic tumours.

In a most preferred embodiment there is provided a method for the treatment and/or prophylaxis of a condition characterised by unwanted TNF-mediated cellular apoptosis, said method comprising administering to said mammal an effective amount of an agent for a time and under conditions sufficient to downregulate the functional level of Bim in an apoptotic or pre-apoptotic cell.

In another preferred embodiment there is provided a method for the treatment and/or prophylaxis of a condition characterised by unwanted soluble TNF-mediated cellular apoptosis, said method comprising administering to said mammal an effective amount of an agent for a time and under conditions sufficient to downregulate the functional levels of Bim and Bid in an apoptotic or pre-apoptotic cell.

In yet another preferred embodiment there is provided a method for the treatment and/or prophylaxis of a condition characterised by unwanted soluble TNF-mediated cellular apoptosis, said method comprising administering to said mammal an effective amount of an agent for a time and under conditions sufficient to downregulate the functional level of Bid in an apoptotic or pre-apoptotic cell.

Preferably, said cellular apoptosis is apoptosis of a cell susceptible to TNF-mediated apoptosis. More preferably said cellular apoptosis is hepatocyte, macrophage or fibroblast apoptosis.

Still more preferably, said cellular apoptosis is pathological hepatocyte apoptosis and said condition is characterised by hepatocyte destruction.

Preferably, said TNF is TNF-α or TNF-β.

Still more preferably, said TNF is TNF-α and:
(i) where said TNF-α is soluble said method comprises downregulating the functional level of Bim and Bid;
(ii) where said TNF-α is membrane-bound said method comprises downregulating the functional level of Bim alone.

In accordance with this aspect of the present invention, in one embodiment said condition is characterised by unwanted apoptosis which is mediated by membrane-bound TNF. Preferably, said condition is autoimmune disease, graft-vs.-host disease, transplant rejection or viral infection, in particular Hepatitis B or Hepatitis C. In another embodiment, said condition is characterised by unwanted apoptosis which is mediated by soluble TNF. In accordance with this embodiment, said conditions are preferably inflammation, sepsis and septic shock.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable non-toxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, Bim or Bid may be administered together with an agonistic agent in order to enhance its effects. Alternatively, in the case of autoimmune induced apoptosis, the Bim or Bid antagonist may be administered together with immunosuppressive drugs. By “sequential” administration is meant a time difference of from seconds, minutes, hours or days between the administration of the two types of molecules. These molecules may be administered in any order.

Another aspect of the present invention relates to the use of an agent capable of modulating the functional level of Bim in an apoptotic or pre-apoptotic cell in the manufacture of a medicament for the treatment and/or prophylaxis of a condition characterised by aberrant TNF-mediated cellular apoptosis in a mammal wherein upregulating said level facilitates the induction of TNF-mediated cellular apoptosis and downregulating said level inhibits or reduces TNF-mediated cellular apoptosis.

Preferably, said cellular apoptosis is apoptosis of a cell susceptible to TNF-mediated apoptosis. More preferably said cellular apoptosis is hepatocyte, macrophage or fibroblast apoptosis. Even more preferably said cellular apoptosis is hepatocyte apoptosis and most preferably pathological hepatocyte apoptosis.

More particularly, there is provided the use of an agent capable of modulating the functional level of Bim in an apoptotic or pre-apoptotic hepatocyte in the manufacture of a medicament for the treatment and/or prophylaxis of a condition characterised by aberrant TNF-mediated hepatocyte apoptosis in a mammal wherein upregulating said level facilitates the induction of TNF-mediated cellular apoptosis and downregulating said level inhibits or reduces TNF-mediated cellular apoptosis.

In a most preferred embodiment there is provided the use of an agent capable of downregulating the functional level of Bim in an apoptotic or pre-apoptotic cell in the manufacture of a medicament for the treatment and/or prophylaxis of a condition characterised by unwanted soluble or membrane-bound TNF-mediated cellular apoptosis.

In another preferred embodiment there is provided the use of an agent capable of downregulating the functional levels of Bim and Bid in an apoptotic or pre-apoptotic cell in the manufacture of a medicament for the treatment and/or prophylaxis of a condition characterised by unwanted soluble TNF-mediated cellular apoptosis.

In yet another preferred embodiment there is provided the use of an agent capable of downregulating the functional levels of Bid within a apoptotic or pre-apoptotic cell in the manufacture of a medicament for the treatment and/or prophylaxis of a condition characterised by unwanted soluble TNF-mediated cellular apoptosis.

Preferably, said cellular apoptosis is apoptosis of a cell susceptible to TNF-mediated apoptosis. More preferably said cellular apoptosis is hepatocyte, macrophage or fibroblast apoptosis. Even more preferably said cellular apoptosis is hepatocyte apoptosis and most preferably pathological hepatocyte apoptosis.

More preferably, said TNF is TNF-α or TNF-β.

Still more preferably, said TNF is TNF-α and:
(i) where said TNF-α is soluble TNF-α said method comprises downregulating the functional levels of Bim and Bid;
(ii) where said TNF-α is membrane-bound TNF-α said method comprises downregulating the functional level of Bim alone.

In accordance with this aspect of the present invention, in one embodiment said condition is characterised by unwanted apoptosis which is mediated by membrane-bound TNF. Preferably, said condition is autoimmune disease, graft-vs.-host disease, transplant rejection or viral infection, in particular Hepatitis B or Hepatitis C. In another embodiment, said condition is characterised by unwanted apoptosis which is mediated by soluble TNF. In accordance with this embodiment, said conditions are preferably inflammation, sepsis and septic shock.

The term “mammal” and “subject” as used herein includes humans, primates, livestock animals (e.g. sheep, pigs, cattle, horses, donkeys), laboratory test animals (e.g. mice, rabbits, rats, guinea pigs), companion animals (e.g. dogs, cats) and captive wild animals (e.g. foxes, kangaroos, deer). Preferably, the mammal is human or a laboratory test animal Even more preferably, the mammal is a human.

In yet another further aspect, the present invention contemplates a pharmaceutical composition comprising the modulatory agent as hereinbefore defined and one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding Bim or Bid or a modulatory agent as hereinbefore defined. The vector may, for example, be a viral vector.

Yet another aspect of the present invention relates to the agent as hereinbefore defined, when used in the method of the invention.

Another aspect of the present invention provides a method for detecting an agent capable of modulating TNF-mediated cellular apoptosis by modulating Bim or Bid functionality said method comprising contacting a cell or extract thereof containing Bim or Bid or its functional equivalent or derivative with a putative agent and detecting an altered expression phenotype.

Reference to “Bim” and “Bid” should be understood as a reference to either the Bim or Bid expression product or to a portion or fragment of the Bim or Bid molecule, such as the Bim or Bid binding regions of these molecules. In this regard, the Bim or Bid expression product is expressed in a cell. The cell may be a host cell which has been transfected with Bim or Bid nucleic acid molecule or it may be a cell which naturally contains the Bim or Bid gene. Reference to “extract thereof” should be understood as a reference to a cell free transcription system.

Reference to detecting an “altered expression phenotype” should be understood as the detection of cellular changes associated with modulation of the interaction of Bim or Bid with its ligands. These may be detectable, for example, as intracellular changes or changes observable extracellularly. For example, this includes, but is not limited to, detecting changes in downstream product levels or functional activities (e.g. mitochondrial respiration).

In a preferred embodiment, the present invention provides a method for detecting an agent capable of modulating TNF-mediated cellular apoptosis by modulating Bim or Bid functionality said method comprising contacting a cell containing said Bim or Bid or its functional equivalent or derivative with a putative agent and detecting an altered apoptosis profile.

The present invention is further described by reference to the following non-limiting examples.

### Example 1

### Materials and Methods

### Mice

Bid deficient mice on an inbred C57BL/6 background were generated. TRAIL^{-/-} (Cretney et al., J Immunol 168:1356-1361, 2002) and bim^{-/-} mice (Bouillet et al., Science 286:1735-1738, 1999) were generated by homologous recombination in 129SV-derived ES cells and have been backcrossed for >10 generations onto the C57BL/6 background. TNF^{-/-} mice (generated using C57BL/6 ES cells) (Korner et al., Eur J Immunol 27:2600-9, 1997) were obtained from Dr. Heinrich Korner, the Centenary Institute of Cancer Medicine and Cell Biology, Sydney, Australia. C57BL/6 pfp^{-/-} mice (Kägi et al., Nature 369:31-37, 1994) were generated using C57BL/6 ES cells. C57BL/6 GrzABM^{-/-} mice were established by intercrossing C57BL/6 GrzM^{-/-} mice with C57BL/6 GrzAB^{-/-} mice (Pao et al., J Immunol 175:3235-43, 2005) (both strains generated using 129Sv ES cells and backcrossed for >12 and 8 generations, respectively). Mice lacking caspase-8 selectively in hepatocytes were generated by crossing mice with a loxP targeted caspase-8 gene, generated on a mixed C57BL/6×129SV background and crossed with C57BL/6 mice for 3 generations (Salmena et al., Genes and Development 17:883-895, 2003), with transgenic mice expressing the Cre recombinase under control of the hepatocyte-specific albumin promoter (backcrossed with C57BL/6 mice for 6 generations). The bid^{-/-}bim^{-/-} mice were generated by serially intercrossing the two parental strains. All experiments with mice were performed according to the guidelines of the animal ethics committees of the Melbourne Health Research Directorate, the Peter MacCallum Cancer and the Ontario Cancer Institute.

### In Vivo Models of Fulminant Hepatitis

For the Fas-mediated hepatitis model, mice were injected intravenously (i.v.) with 0.25 μg/g body weight recombinant soluble Fas ligand (FLAG® tagged, Apotech) that had been crosslinked with 2 μg anti-FLAG® antibody (M2, SIGMA) per μg of FasL. For the T-cell activation mediated hepatitis, mice were injected i.v. with 30 μg/g body weight of ConA (SIGMA). For the LPS model, mice were injected intraperitoneally (i.p.) with 10, 100 or 1000 ng of LPS (DIFCO) in the presence of 20 mg of the liver transcriptional inhibitor D-(+)-galactosamine (GalN, SIGMA). At the time when wt mice succumbed to any of these treatments, all mice of an experimental group were sacrificed, bled (for serum analysis of liver enzymes) and the livers surgically removed for histological analysis. Statistical analyses were performed applying a two-tailed unpaired t test.

### Western Blotting

Mouse livers were surgically removed and cell suspensions prepared by passing them through a stainless steel sieve. Red blood cells were lysed in a hypotonic buffer and hepatocyte lysates prepared in a buffer containing 20 mM Tris/HCl pH 7.4, 135 Mm NaCl, 1.5 mM MgCl₂, 1 mM EGTA, 1% Triton X-100, 10% glycerol, 500 μg/mL Pefabloc (AEBSF), 1 μg/mL each of Leupeptin, Aprotinin and Pepstatin, 100 μg/mL soybean trypsin inhibitor and 2 μg/mL E64. Proteins (40 μg) in cell lysates were size-separated on precast 12% SDS PAGE gradient gels (Invitrogen). Membranes were probed with a rat anti-Bid monoclonal antibody (clone 2D1; TK, David C S Huang and AS submitted), a polyclonal rabbit anti-Bim antibody (Stressgen) or a mouse monoclonal antibody to caspase-7 (gift from Y. Lazebnik). Probing with a mouse monoclonal antibody to β-actin (SIGMA, AC-40) served as loading control.

### Results

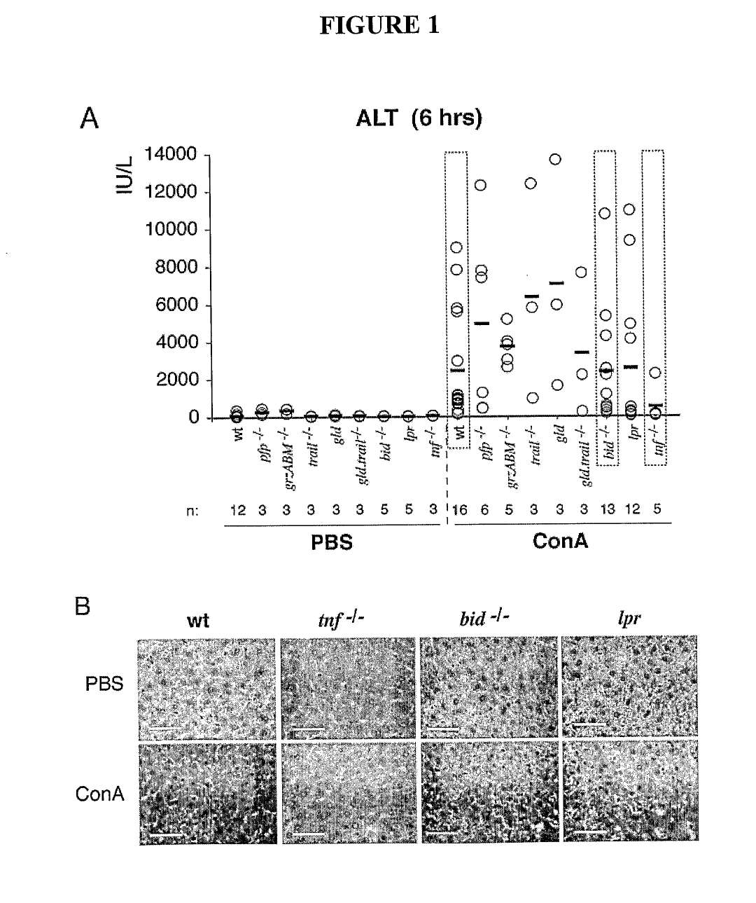

Injection of the lectin ConA into mice causes systemic activation of T lymphocytes that results in fatal hepatitis (Tiegs et al., J Clin Invest 90:196-203, 1992). A collection of gene-targeted (TNF-, TRAIL-, perforin-, granzymes A,B,M-deficient) or the spontaneous fas mutant lpr mice (all on an inbred or >8× backcrossed C57BL/6 genetic background) were analysed and it was found that only TNF-deficiency provided resistance to this treatment (FIG. 1A). All other animals were moribund within 6-8 hr of ConA injection, presenting with elevated serum ALT levels and histological evidence of severe hepatocyte destruction (FIG. 1A). Bid^{-/-} mice responded to this treatment in a manner indistinguishable from wt animals. All succumbed within 6-8 hr post-injection and exhibited no significant difference in ALT serum levels or liver histopathology compared to wt animals (FIGS. 1A and 1B).

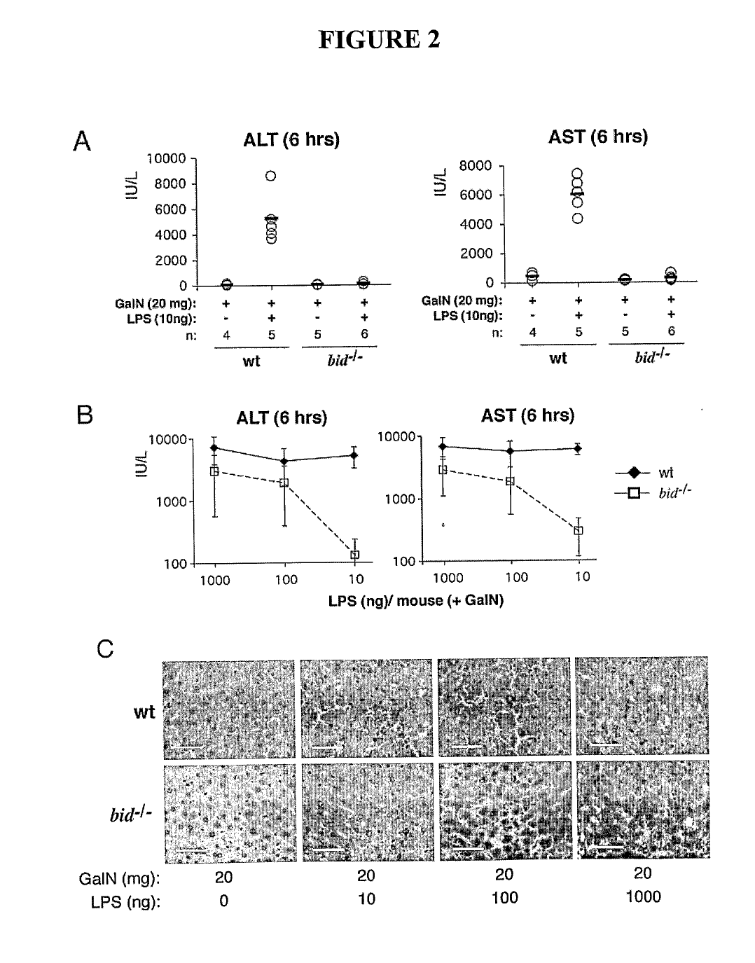

Intraperitoneal (i.p.) injection of low doses of bacterial LPS in the presence of the liver-specific transcriptional inhibitor D-(+)-galactosamine (GalN) rapidly causes fatal hepatocyte destruction, accompanied by severe liver pathology and elevated serum levels of ALT and AST (Galanos et al., Proc Natl Acad Sci USA 76:5939-5943, 1979). Upon injection of very low doses of LPS (10 ng per mouse) plus GalN, bid^{-/-} mice were less severely affected than wt animals. All wt mice succumbed within 6-8 hr of treatment, whereas all bid^{-/-} mice remained alive at that time point, presenting with significantly lower ALT and AST serum levels (FIG. 2A; bid^{-/-} vs wt: p<0.0002 for ALT, p<0.0001 for AST) and less severe liver destruction (FIG. 2C). However, only 3 out of 7 bid^{-/-} mice survived this treatment for 5 days or more, the other 4 became moribund within 24 hours. Moreover, when the dose of LPS was increased to 100 or 1000 ng, all wt and bid^{-/-} animals died at 6-8 hr post-injection, but the serum levels of ALT and AST were still significantly lower in the bid^{-/-} mice compared to the wt littermates (FIG. 2B; p ≦0.02 for ALT and p ≦0.002 for AST). Histological examination of liver sections taken after 6 hr showed less hepatocyte damage in bid^{-/-} mice compared to wt controls for all concentrations of LPS (FIG. 2C).

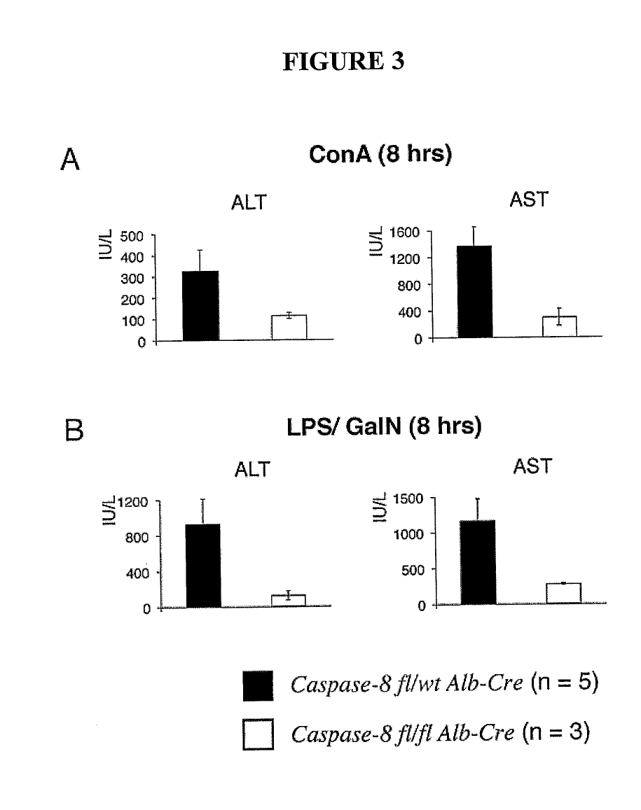

Mice lacking caspase-8 selectively in hepatocytes (caspase-8 loxP homozygotes expressing the Cre recombinase under control of the hepatocyte-specific albumin promoter) were used to investigate whether this protease is required for TNF-mediated killing of hepatocytes in vivo. Upon challenge with ConA or LPS plus GalN, all littermate controls succumbed within 6-8 hr, presenting at autopsy with abnormally elevated serum levels of ALT and AST (FIG. 3) and extensive disruption of liver architecture. In contrast, all mice lacking caspase-8 in hepatocytes survived these treatments and showed only minor elevation of serum ALT and AST levels (FIG. 3; caspase-8 deficient vs control mice, for LPS+GalN: p<0.015 for ALT, p<0.0015 for AST; for ConA: p<0.0035 for both ALT and AST) and retained normal liver structure.

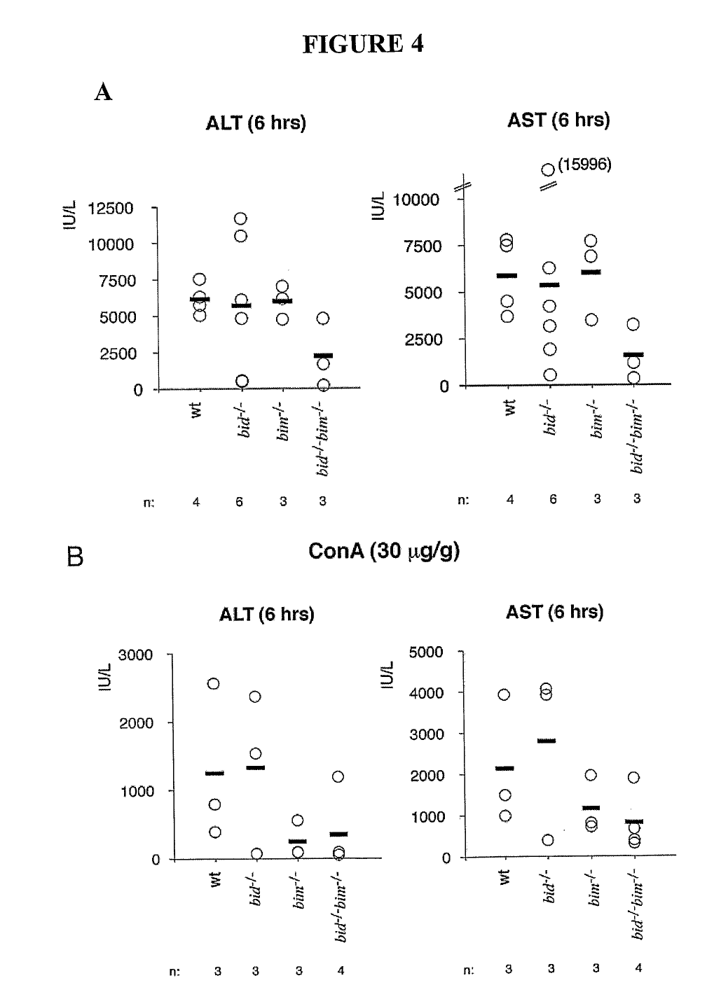

Although bim^{-/-} mice were normally sensitive to injection with LPS (100 ng) plus GalN, Bid/Bim double knock-out mice were resistant to this treatment. At the time when all wt, bid^{-/-} and bim^{-/-} mice were moribund, all bid^{-/-}bim^{-/-} mice still appeared healthy and their serum ALT and AST levels were significantly lower than those found in the control animals (FIG. 4A; bid^{-/-} bim^{-/-} vs wt mice, p<0.03 for ALT, p<0.035 for AST). Histological analysis confirmed reduced liver destruction in bid^{-/-}bim^{-/-} mice compared to the control animals. Synergy between loss of Bid and Bim appeared to be specific to LPS+GalN-induced (TNF-mediated) cell killing, since fibroblasts and T lymphocytes from bid^{-/-}bim^{-/-} mice were normally sensitive to apoptosis induced by DNA damage or certain other cytotoxic insults.

The role of Bim and Bid plus Bim in ConA induced hepatitis was also examined. After 8 hr of exposure to ConA when all wt or bid^{-/-} mice were moribund, all animals lacking Bim appeared much healthier and presented with considerably reduced serum levels of ALT and AST (FIG. 4B) and less destruction of liver architecture. The bid^{-/-} bim^{-/-} and the bim^{-/-} mice responded indistinguishably (FIG. 4B) indicating that Bim is the major BH3-only protein involved in ConA-induced hepatocyte killing.

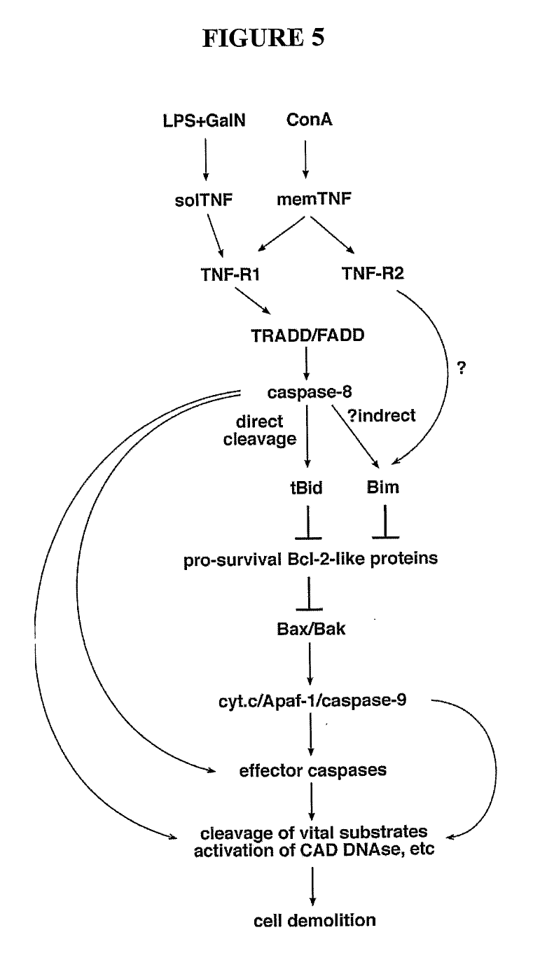

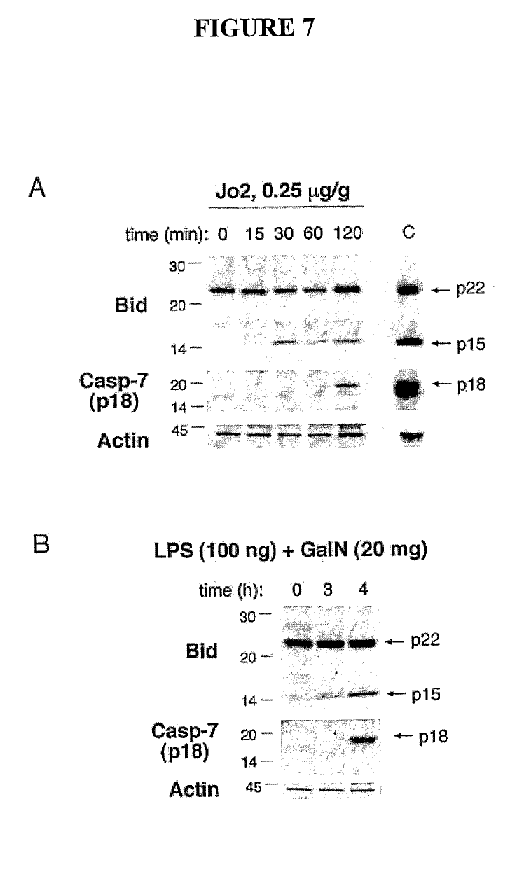

Using two distinct experimental models, the roles of caspase-8 and pro-apoptotic BH3-only Bcl-2 family members in TNF-mediated liver pathology were defined. Hepatocyte killing caused by extensive (polyclonal) T cell activation, which is mediated by membrane-bound TNF, signalling through both TNF-R1 and TNF-R2 (Dusters et al., 1997, supra; Trautwein et al., 1998, supra), or that caused by LPS plus GalN, which instead is mediated by soluble TNF-TNF-R1 signalling (Grivennikov et al., 2005, supra), both required caspase-8. This indicates that these two distinct forms of TNF trigger hepatocyte apoptosis by highly similar mechanisms. There are, however, differences between membrane-bound and soluble TNF-induced apoptosis, since loss of Bim protected mice against ConA-induced hepatitis and Bid-deficiency afforded some, albeit limited, protection against injection of LPS plus GalN. This indicates that TNF-R2 stimulation, which is engaged most efficiently by membrane-bound TNF (Grell et al., Cell 83:793-802, 1995), activates a signal that renders this response heavily dependent on Bim but not Bid (FIG. 5). In contrast, soluble TNF-TNF-R1 induced hepatocyte killing requires not only Bim but also Bid (FIG. 5). It was found that injection with anti-Fas antibody, which requires tBid for hepatocyte killing (Yin et al., 1999, supra), elicits less caspase-8 activity (as judged by processing of Bid and caspase-7) than injection with LPS (100 ng) plus GalN, which kills by a mostly Bid-independent process (FIG. 7).

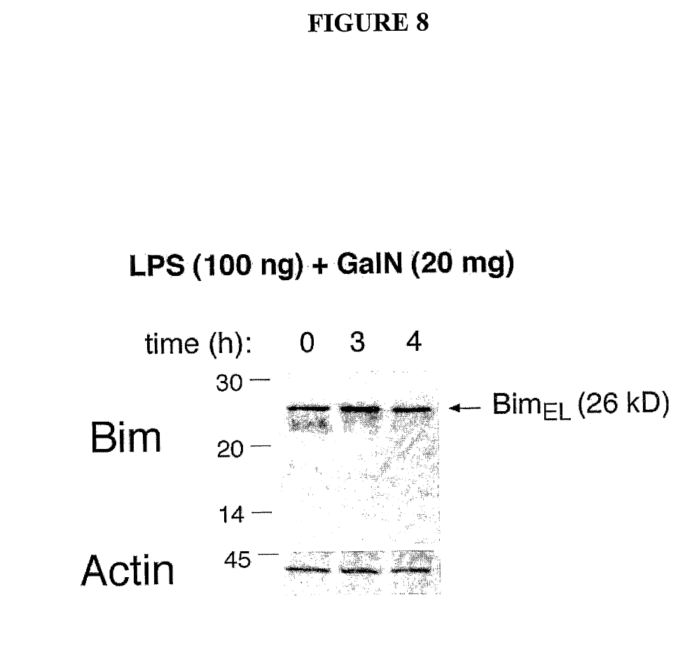

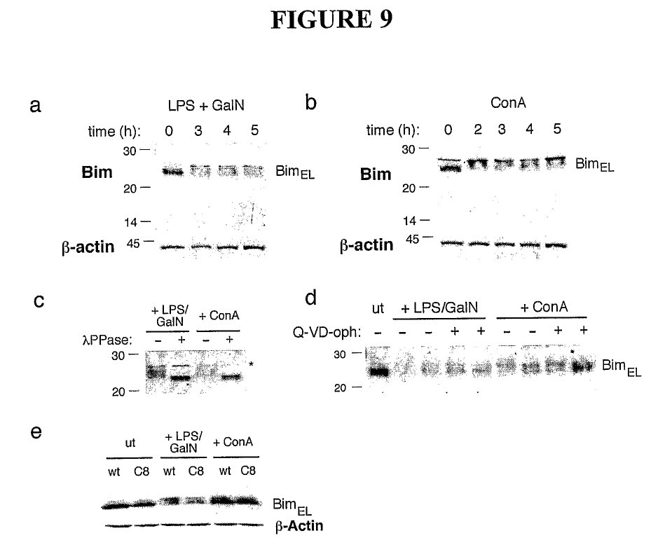

It appears that Bim, like tBid, can function as an amplifier of the apoptotic cascade. Bim may do this through inhibition of pro-survival Bcl-2 family members, leading to Bax/Bak-dependent activation of caspase-9 and effector caspases (FIG. 5). Although caspase-mediated activation of Bim_{EL} (the most abundantly expressed isoform of Bim in the liver and other tissues (O'Reilly et al., Am J Pathol 157:449-461, 2000) has been reported (Chen & Zhou, 2004, supra) there was no evidence found for Bim_{EL} cleavage in livers of ConA or LPS plus GalN injected mice (FIG. 8). It is therefore possible that caspase-8 activates Bim indirectly. Western Blot analysis of liver extracts from mice treated with LPS+GalN or ConA demonstrates that there is a rapid occurrence of a post-translational modification of Bim (FIG. 9b). Treatment with λPPase indicates that this post-translational modification is a phosphorylation (FIG. 9c) and occurs independently of caspase 8 (FIG. 9e) or other caspases.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Aggarwal, B. B. Signalling pathways of the TNF superfamily: a double-edged sword. Nat Rev Immunol 3:745-756 (2003).
Arch et al., Genes & Devel. 12:2821 (1998)
Bouillet, P. et al. Proapoptotic Bcl-2 relative Bim required for certain apoptotic responses, leukocyte homeostasis, and to preclude autoimmunity. Science 286:1735-1738 (1999).
Bunin B. A., Plunkett M. J. and Ellman T. A. (1994) The combinatorial synthesis and chemical and biological evaluation of a 1,4-benzodiazepine library. Proc. Natl. Acad. Sci. USA, 91:4708-4712
Chen, D. & Zhou, Q. Caspase cleavage of BimEL triggers a positive feedback amplification of apoptotic signaling. Proc Natl Acad Sci USA 101:1235-1240 (2004).
Chinnaiyan et al., Science 274:990 (1996)
Cretney, E. et al. Increased susceptibility to tumor initiation and metastasis in TNF-related apoptosis-inducing ligand-deficient mice. J Immunol 168:1356-1361 (2002).
DeWitt S. H., Kiely J. S., Stankovic C. J., Schroeder M. C., Cody D. M. and Pavia M. R. (1993) “Diversomers”: an approach to nonpeptide, nonoligomeric chemical diversity. Proc. Natl. Acad. Sci. USA, 90:6909-6913
Galanos et al., Galactosamine-induced sensitization to the lethal effects of endotoxin. Proc Natl Acad Sci USA 76:5939-5943 (1979).
Grell, M. et al. The transmembrane form of tumor necrosis factor is the prime activating ligand of the 80 kDa tumor necrosis factor receptor. Cell 83:793-802 (1995).
Grivennikov, S. I. et al. Distinct and nonredundant in vivo functions of TNF produced by t cells and macrophages/neutrophils: protective and deleterious effects. Immunity 22:93-104 (2005).
Huang, D. C. S. & Strasser, A. BH3-only proteins—essential initiators of apoptotic cell death. Cell 103:839-842 (2000).
Kägi, D. et al. Cytotoxicity mediated by T cells and natural killer cells is greatly impaired in perforin-deficient mice. Nature 369:31-37 (1994).
Kang, T. B. et al. Caspase-8 serves both apoptotic and nonapoptotic roles. J Immunol 173:2976-2984 (2004).
Korner, H. et al. Distinct roles for lymphotoxin-alpha and tumor necrosis factor in organogenesis and spatial organization of lymphoid tissue. Eur J Immunol 27:2600-9 (1997).
Kusters, S. et al. In vivo evidence for a functional role of both tumor necrosis factor (TNF) receptors and transmembrane TNF in experimental hepatitis. Eur J Immunol 27:2870-2875 (1997).
Li et al., Cleavage of BID by caspase 8 mediates the mitochondrial damage in the Fas pathway of apoptosis. Cell 94:491-501 (1998).
Locksley et al., Cell 104:487 (2001)
Luo et al., Bid, a Bcl-2 interacting protein, mediates cytochrome c release from mitochondria in response to activation of cell surface death receptors. Cell 94:481-490 (1998).
Nowak, M. et al. LPS-induced liver injury in D-galactosamine-sensitized mice requires secreted TNF-alpha and the TNF-β55 receptor. Am J Physiol Regul Integr Comp Physiol 278:R1202-R1209 (2000).
O'Reilly, L. A. et al. The proapoptotic BH3-only protein bim is expressed in hematopoietic, epithelial, neuronal, and germ cells. Am J Pathol 157:449-461 (2000).
Pao, L. I. et al. Functional analysis of granzyme M and its role in immunity to infection. J Immunol 175:3235-43 (2005).
Pfeffer, K. et al. Mice deficient for the 55 kd tumor necrosis factor receptor are resistant to endotoxic shock, yet succumb to L. monocytogenes infection. Cell 73:457-467 (1993).
Puthalakath et al., The pro-apoptotic activity of the Bcl-2 family member Bim is regulated by interaction with the dynein motor complex. Mol Cell 3:287-296 (1999).
Puthalakath H, Strasser A., Keeping killers on a tight leash: transcriptional and post-translational control of the pro-apoptotic activity of BH3-only proteins. Cell Death Differ. 2002, 9(5):505-12
Rothe, J. et al. Mice lacking the tumour necrosis factor receptor 1 are resistant to TNF-mediated toxicity but highly susceptible to infection by Listeria monocytogenes. Nature 364:798-802 (1993).
Salmena, L. et al. Essential role for caspase 8 in T-cell homeostasis and T-cell-mediated immunity. Genes and Development 17:883-895 (2003).
Tagawa et al., Involvement of Fas/Fas ligand system-mediated apoptosis in the development of concanavalin A-induced hepatitis. European Journal of Immunology 28:4105-4113 (1998).
Tartaglia et al., Cell 73:213 (1993)
Tiegs et al., A T cell-dependent experimental liver injury in mice inducible by concanavalin A. J Clin Invest 90:196-203 (1992).
Trautwein, C. et al. Concanavalin A-induced liver cell damage: activation of intracellular pathways triggered by tumor necrosis factor in mice. Gastroenterology 114:1035-1045 (1998).
Wang et al., BID: a novel BH3 domain-only death agonist. Genes and Development 10:2859-2869 (1996).
Watanabe et al., Concanavalin A induces perforin-mediated but not Fas-mediated hepatic injury. Hepatology 24:702-710 (1996).
Yin, X.-M. et al. Bid-deficient mice are resistant to Fas-induced hepatocellular apoptosis. Nature 400:886-891 (1999).
Zha et al., Posttranslational N-myristoylation of BID as a molecular switch for targeting mitochondria and apoptosis. Science 290:1761-1765, (2000).
Zheng et al., Critical roles of TRAIL in hepatic cell death and hepatic inflammation. J Clin Invest 113:58-64 (2004).

1. A method of modulating mammalian TNF-mediated cellular apoptosis in vivo or in vitro, said method comprising:
a) modulating the functional level of Bim in an apoptotic or pre-apoptotic cell, wherein said TNF is membrane bound; or
b) modulating the functional level of Bim, Bid or Bim and Bid in an apoptotic or pre-apoptotic cell, wherein said TNF is soluble;
wherein upregulating said level facilitates the induction of TNF-mediated cellular apoptosis and downregulating said level inhibits or reduces TNF-mediated cellular apoptosis.

2. (canceled)

3. A method for the treatment and/or prophylaxis of a condition characterized by aberrant TNF-mediated cellular apoptosis in a mammal, said method comprising:
a) modulating the functional level of Bim in an apoptotic or pre-apoptotic cell in said mammal, wherein said TNF is membrane bound; or
b) modulating the functional level of Bim, Bid or Bim and Bid in an apoptotic or pre-apoptotic cell in said mammal, wherein said TNF is soluble;
wherein upregulating said level facilitates the induction of TNF-mediated cellular apoptosis and downregulating said level inhibits or reduces TNF-mediated cellular apoptosis.

4. (canceled)

5. The method according to claim 1 or 3 wherein said TNF is soluble and the functional levels of Bim and Bid are modulated.

6. The method according to claim 1 or 3 wherein said cellular apoptosis is pathological apoptosis.

7. The method according to claim 6 wherein said cell is a hepatocyte, macrophage or fibroblast.

8. The method according to claim 7 wherein said cell is a hepatocyte.

9. The method according to claim 6 wherein said TNF is TNFα or TNFβ.

10. The method according to claim 6 wherein said TNF is TNFα.

11. The method according to claim 9 wherein said modulation is upregulation and said upregulation is achieved by introducing into said cell (i) a nucleic acid molecule encoding Bim and/or Bid or functional fragment or derivative thereof or (ii) the Bim and/or Bid expression product or functional fragment or derivative thereof.

12. The method according to claim 9 wherein said modulation is upregulation and said upregulation is achieved by introducing into said cell a proteinaceous or non-proteinaceous molecule which functions as an agonist of Bim.

13. The method according to claim 12 wherein said agonist is a kinase or phosphatase which acts to inhibit sequestration of Bim to the microtubule-associated dynein motor complex, prevent degradation of Bim or promotes translocation of Bim to mitochondria.

14. The method according to claim 13 wherein said agonist is INK kinase or protein phosphatase 2A.

15. The method according to claim 12 wherein said agonist acts to stimulate the interaction of Bid with other members of the bcl-2 family.

16. The method according to claim 12 wherein said agonist is a BH3 mimetic.

17. The method according to claim 9 wherein said modulation is upregulation and said upregulation is achieved by introducing into said cell a proteinaceous or non-proteinaceous molecule which functions as an agonist of Bid.

18. The method according to claim 17 wherein said agonist is a caspase, granzyme, cathepsin or calpain.

19. The method according to claim 18 wherein said caspase is caspase 8 or 10.

20. The method according to claim 17 wherein said agonist acts to stimulate the interaction of Bid with other members of the bcl-2 family.

21. The method according to claim 9 wherein said modulation is downregulation and said downregulation is achieved by introducing into said cell a proteinaceous or non-proteinaceous molecule which functions as an antagonist of Bim and/or Bid.

22. The method according to claim 21 wherein said antagonist is an antibody directed to the Bim and/or Bid protein or nucleic acid molecule.

23. The method according to claim 21 wherein said antagonist is an RNA aptamer directed to Bim and/or Bid.

24. The method according to claim 21 wherein said antagonist is an antagonist of Bid and is an inhibitor of a caspase, granzyme, cathepsin or calpain.

25. The method according to claim 24 wherein said caspase is caspase 8 or 10.

26. The method according to claim 21 wherein said antagonist is an antagonist of Bim and acts to promote sequestration of Bim to the microtubule-associated dynein motor complex, promote degradation of Bim or prevent translocation of Bim to mitochondria.

27. The method according to claim 26 wherein said antagonist is ERK kinase.

28. The method according to claim 9 wherein said modulation is achieved by introducing into said cell a proteinaceous or non-proteinaceous molecule which modulates transcriptional and/or translational regulation of a Bim nucleic acid molecule.

29. The method according to claim 28 wherein said molecule is a FOXO transcription factor.

30. The method according to claim 28 wherein said molecule is an antisense molecule directed to Bim and/or Bid DNA or RNA.

31. The method according to claim 30 wherein said molecule is a ribozyme.

32. The method according to claim 3 wherein said condition is unwanted cellular apoptosis and said modulation is downregulation of the functional level of Bim and/or Bid.

33. The method according to claim 32 wherein said cellular apoptosis is hepatocyte, macrophage or fibroblast apoptosis.

34. The method according to claim 3 wherein said condition is an autoimmune disease, graft-vs-host disease, transplant rejection or a viral infection.

35. The method according to claim 3 wherein said condition is hepatitis, alcoholic liver disease or hepatocyte destruction.

36. The method according to claim 35 wherein said hepatitis is viral hepatitis or hepatitis caused by a drug overdose.

37. The method according to claim 34 wherein said viral infection is by Hepatitis B or Hepatitis C.

38. The method according to claim 3 wherein said condition is inflammation, sepsis or septic shock.

39. The method according to claim 3 wherein said aberrant apoptosis is inadequate apoptosis and said modulation is upregulation of the functional level of Bim and/or Bid.

40. The method according to claim 39 wherein said condition is a hepatic tumor.

41.-78. (canceled)
